(19) United States
(12) Patent Application Publication
(10) Pub. No.: US 2010/0159547 A1
(43) Pub. Date: Jun. 24, 2010

(54) METHOD FOR SEPARATING OIL FROM AN OIL CONTAINING MATERIAL, METHOD FOR THE PRODUCTION OF ETHANOL, AND ETHANOL PRODUCTION FACILITY

(75) Inventors: James Faulconbridge, Maplewood, MN (US); Robert Wills, Brooklyn Park, MN (US)

Correspondence Address: MERCHANT & GOULD PC, P.O. BOX 2903, MINNEAPOLIS, MN, 55402-0903, US

(73) Assignee: KFI Intellectual Properties L.L.C., Saint Paul, MN (US)

(21) Appl. No.: 12/622,543

(22) Filed: Nov. 20, 2009

Related U.S. Application Data
(60) Provisional application No. 61/199,875, filed on Nov. 20, 2008.
(60) Provisional application No. 61/233,763, filed on Aug. 13, 2009.

(51) Int. Cl. C12P 7/06; C11B 1/10

(52) U.S. Cl. 435/161; 554/8

(57) ABSTRACT

A method for separating oil from flour includes steps of: separating flour into a first stream comprising coarse flour and a second stream comprising fine flour; combining a first extraction solvent with the first stream and extracting oil from the first stream to provide a first miscella and coarse solids; combining a second extraction solvent with the second stream and extracting oil from the second stream to form a second miscella and fine solids; and recovering oil from the first miscella and the second miscella. Another method that can be used separate from or in combination with the previously described method includes steps of: extracting oil from the oil containing, non-fermented, vegetable material with a water immiscible extraction solvent and forming a mixture; combining the mixture with water and forming an organic phase and a water phase; and separating the organic phase and the water phase to form: the organic phase comprising the water and oil enriched immiscible extraction solvent; and the water phase comprising water and oil depleted, non-fermented, vegetable material. A method for producing ethanol and an ethanol production facilitate are described.

### CROSS REFERENCE TO RELATED APPLICATIONS

This application claims priority, to the extent applicable, to U.S. Provisional Application Ser. No. 61/199,875 that was filed with the United States Patent and Trademark Office on Nov. 20, 2008 and U.S. Provisional Application Ser. No. 61/233,763 that was filed with the United States Patent and Trademark Office on Aug. 13, 2009. The entire disclosures of U.S. Provisional Ser. No. 61/199,875 and U.S. Provisional Application Ser. No. 61/233,763 are incorporated in their entirety.

### FIELD OF THE DISCLOSURE

The present disclosure is directed to a method for the separation of oil from an oil containing material, a method for the production of ethanol, and an ethanol production facility.

### BACKGROUND

It is often desirable to recover oil from an oil containing material. In some cases, oil containing material can be pressed for the recovery of crude oil. In other cases, the oil containing material can be contacted with an extraction solvent that solubilizes the oil, and the oil can be separated from the solvent. Even materials that have been pressed for the recovery of crude oil can be contacted with an extraction solvent for the recovery of additional oil.

Oil containing materials that are typically processed for the recovery of oil include vegetable material. Vegetable material refer to plants or plant products and can include, for example, grains and seeds. Often, the vegetable material is ground, cracked, milled or otherwise processed to increase its surface area. The processed vegetable material is then contacted with an extraction solvent for the recovery of oil.

There exist oil containing materials that also contain a sugar source, and those materials are often subjected to fermentation for the production of ethanol. Corn is an exemplary grain that is commonly subjected to fermentation for the production of ethanol. Because corn contains valuable corn oil, it is often desirable to recover the corn oil either prior to fermentation or after fermentation. Various techniques for the recovery of oil can occur in either a wet milling process or a dry milling process.

In the dry milling process, the corn kernels are crushed, flaked, milled, or otherwise modified into smaller pieces than the whole kernel. Because the majority of the corn oil is found in the germ of the kernel, the germ is often separated from the rest of the kernel. An extraction solvent is then passed over, through, and otherwise around the corn pieces, and at least a portion of the oil becomes solubilized in the extraction solvent. The resulting extraction solvent and oil can be separated from the solids by filtration. The oil is then recovered by vaporizing the extraction solvent.

Efforts have been made for the recovery of oil from seed germ prior to fermentation. For example, see Cao et al., Enzymatic Hydrolysis of Corn Starch After Extraction of Corn Oil with Ethanol, Applied Biochemistry and Biotechnology, Vol. 57/58, pages 39-47, 1996, and Hojilla-Evangelist et al., Sequential Extraction Processing of Flaked Whole Corn: Alternative Corn Fractionation Technology for Ethanol Production, Cereal Chemistry, 1992.

### SUMMARY

A method for separating oil from flour is described. The method includes steps of: separating flour into a first stream comprising coarse flour and a second stream comprising fine flour; combining a first extraction solvent with the first stream and extracting oil from the first stream to provide a first miscella and coarse solids; combining a second extraction solvent with the second stream and extracting oil from the second stream to form a second miscella and fine solids; and recovering oil from the first miscella and the second miscella.

A method for producing ethanol is described. The method includes steps: separating flour into a first stream comprising coarse flour and a second stream comprising fine flour; combining a first extraction solvent with the first stream and extracting oil from the first stream to provide a first miscella and coarse solids; combining a second extraction solvent with the second stream and extracting oil from the second stream to form a miscella and fine solids; recovering the coarse solids and the fine solids and drying to form dried deoiled solids; and fermenting the dried deoiled solids.

An ethanol production facility is described that includes: a screener for separating flour into a first stream comprising coarse flour and a second stream comprising fine flour; a first extractor for receiving the first stream comprising coarse flour and an extraction solvent for extracting oil from the first stream to provide a first miscella and coarse solids; a first extractor for receiving the first stream comprising fine flour and an extraction solvent for extracting oil from the first stream to provide a first miscella and fine solids; and a centrifuge for separating the second miscella and the fine solids.

A method for separating oil from an oil containing, non-fermented, vegetable material is described. The method includes steps of: extracting oil from the oil containing, non-fermented, vegetable material with a water immiscible extraction solvent and forming a mixture; combining the mixture with water and forming an organic phase and a water phase; and separating the organic phase and the water phase to form: (a) the organic phase comprising the water and oil enriched water immiscible extraction solvent; and (b) the water phase comprising water and oil depleted, non-fermented, vegetable material. The method for separating oil from an oil containing, non-fermented, vegetable material can be practiced separately from or in combination with the previously described method for separating oil from flour. For example, the method can be practiced on the first stream comprising coarse flour, the second stream comprising fine flour, or both the first stream comprising coarse flour and the second stream comprising fine flour.

A method for producing ethanol is described. The method includes steps of: combining an oil containing, non-fermented, vegetable material with a water immiscible extraction solvent and forming crude mixture; combining the crude mixture with water and forming an organic phase and an aqueous phase; separating the organic phase and the aqueous phase; recovering oil from the organic phase; heating the aqueous phase; and fermenting to the aqueous phase to form alcohol.

An ethanol production facility is described that includes: a first mixing vessel for mixing oil containing, non-fermented, vegetable material and a water immiscible extraction solvent to solubilize at least a portion of the oil from the oil containing, non-fermented, vegetable material to form a crude mixture of an oil depleted vegetable material and an oil enriched extraction solvent; a second mixing vessel for mixing water with the crude mixture to form a multiple phase mixture; a phase separation vessel or device for allowing the multiple phase mixture to phase separate into an organic phase and an aqueous phase; and a fermentation vessel for fermenting the aqueous phase, wherein the first mixing vessel, the second mixing vessel, the phase separation vessel, and the fermentation vessel are in fluid connection.

### BRIEF DESCRIPTION OF THE DRAWING

FIG. 1 is a schematic diagram of an alcohol production facility according to the principles of the present invention.

FIG. 2 is a schematic diagram of a fermentation system according to the principles of the present invention.

FIG. 3 is a schematic diagram of a process for separating oil from an oil containing nonfermented vegetable material according to the principles of the present invention.

FIG. 4 is a schematic diagram of an exemplary portion of a dry milling process according to the principles of the present invention.

### DETAILED DESCRIPTION

Oil can be recovered from oil containing material such as flour by extraction with a extraction solvent. After extraction, the extraction solvent resulting from the extraction can be characterized as oil enriched, and the resulting solids material from the extraction can be characterized as oil depleted. The term “oil enriched” means that the material contains more oil as a result of extraction than it contained before extraction. The phrase “oil depleted” means that the material contains less oil as a result of extraction than it contained before to extraction. The phrase “oil depleted” does not mean the material contains no oil. It is expected that in most situations, the oil containing material subjected to extraction will contain some amount of oil after extraction. There is no requirement that the extraction step results in the removal of all of the oil from the oil containing material. In many applications, the extraction can provide removal of at least about 30% of the oil from the oil containing material, and preferably a removal of at least about 50% of the oil from the oil containing material. The extraction can result in the removal of all of the oil from the oil containing material. In many applications, however, it is expected that the extraction will result in the removal of less than about 90% of the oil from the oil containing material. The reference to a percent of oil removal should be understood as a weight percent based on all of the oil present in the oil containing material.

The oil enriched extraction solvent can be separated from the resulting oil depleted material. One technique for separating the oil enriched extraction solvent from the oil depleted material is filtration. For certain types of flour, filtration for the separation of the oil enriched extraction solvent from the oil depleted fine material (fines) can be difficult or problematic because of the tendency of the fine material in the flour to clog the screen or filter in a conventional extractor. In addition, the fine material may bypass the screen or filter in a conventional extractor and thereby accumulate and potentially plug downstream processing equipment such as pumps. In order to handle the presence of fine material in the flour, the flour can be separated into at least two streams, a coarse flour stream and a fine flour stream, prior to extraction and the two streams can be separately extracted. The coarse flour can be subjected to extraction and, because of the size of the particles in the coarse flour, can provide for the drainage of the extraction solvent therefrom without clogging the screen or filter. The fine flour, however, has a tendency to clog the screen or filter in a conventional extractor or bypass the screen or filter and damage downstream processing equipment. Because of the size and distribution of the particulates in the fine material, the combination of oil enriched extraction solvent and oil depleted fine solids is pumpable and can be pumped to a centrifuge for the separation of the oil enriched extraction solvent from the oil depleted fine solids. Also, a portion of the oil enriched solvent can be separated from the combination of oil enriched extraction solvent and oil depleted find solids prior to separation in a centrifuge.

If the resulting oil depleted material contains a source of sugar sufficient for ethanol production, it may be desirable to ferment the oil depleted material for the production of ethanol. The source of sugar can include simple sugars, complex sugars such as starch, or mixtures thereof. By way of example, the source of sugar can include monosaccharides such as glucose and fructose, disaccharides, such as sucrose and lactose, polysaccharides such as starch, and mixtures thereof.

The oil containing material can be provided as “flour.” In general, flour refers to a material that has been reduced in size to form particles. While flour is often prepared as a result of grinding, flour can also be prepared by other techniques that result in a decrease in particle size and an increase in overall surface area. Flour can be considered a dry material. While a dry material is not necessarily free of water, it can feel dry to touch and can flow as a dry material rather than as a slurry. Flour typically has a water content that is sufficiently low so that the flour is shelf stable and does not quickly rot or degrade. Shelf stability typically refers to the ability to store flour, in a dry condition, for at least a couple of months. Typically, the water content of flour is less than about 12 wt. %.

The flour can be provided as a vegetable material. The characterization of the material as “oil containing” means that the material contains a desirable amount of the oil so that the material can be processed for the recovery of the oil. A vegetable material refers to plants or plant products. An oil containing vegetable material refers to plants or plant products that contain oil for recovery. Exemplary oil containing vegetable materials include, for example, grains and seeds. Exemplary oil containing grains include wheat, rice, corn, and oats. In a typical dry mill ethanol plant, the incoming corn kernels are screened for impurities and then run through a hammer mill to break open the corn kernels. The corn product of a hammer mill is often referred to as corn flour.

The flour can be provided as “non-fermented” which means that the material has not been actively fermented to produce alcohol. The reference to “non-fermented” is not intended to exclude material that has experienced fermentation as a result of storage. It is expected that many materials, as a result of being stored for a length of time, may undergo some level of fermentation. Instead, the term “non-fermented” is used to exclude those materials that have been actively subjected to fermentation for the production and recovery of alcohol. Stillage from a fermentation process is an example of a fermented material because it is the solid material resulting from ethanol production. Oil recovered prior to fermentation if often considered to be a higher quality oil compared with oil recovered after fermentation. In the case of recovering oil from corn flour, oil recovered prior to fermentation is often valued higher than oil recovered after fermentation. If desired, the process for recovering oil from flour can be practiced on material after fermentation.

The phrase “extraction solvent” refers to solvents that are capable of solubilizing the oil found in the flour in order to extract oil therefrom. Extraction solvents for extracting oil can include organic solvents that have a tendency to solubilize the oil in order to separate the oil from the flour containing the oil. The extraction solvent is then passed over, through, or otherwise around the flour until at least a portion of the oil becomes solubilized in the extraction solvent. The extraction solvent, when contacted with the flour, has a tendency to solubilize the oil and thereby pull the oil away from the flour. This process is referred to as extraction.

Exemplary extraction solvents for the extraction of oil from flour include alkanes, alkenes, ethers, alcohol, aldehydes, aromatic hydrocarbons, and mixtures thereof. The extraction solvent can be provided as a halogenated form of the organic solvent. Certain solvents are better extraction solvents for certain oils compared with other solvents. The extraction solvent, therefore, can be selected depending on the oil targeted for extraction. Exemplary alkanes that can be used include propane, butane, pentane, methyl pentane, cyclohexane, hexane, and heptanes. Hexane can be a desirable extraction solvent for the extraction of corn oil from corn flour. Exemplary alkenes that can be used include ethylene, propylene, butene, isobutylene, pentene, and hexene. Exemplary ethers that can be used include ethyl ether, dimethyl ether, and isopropyl ether. Exemplary aldehydes that can be used include formaldehyde and glutaraldeyde. Exemplary aromatic hydrocarbons that can be used include benzene, toluene, and xylene. An exemplary halogenated solvent that can be used includes n-propyl bromide. Exemplary alcohols that can be used include alcohols having at least 6 carbon atoms such as, for example, hexanol, heptanol, and octanol.

In the ethanol production industry, materials fermented to produce ethanol typically contain a sufficiently high level of a sugar source so that the material can be economically fermented to produce a high level of ethanol. Starch is an exemplary sugar source found in many vegetable materials useful for production of ethanol. In addition, the materials often contain both oil that is valuable for recovery and a sugar source that can be fermented. An exemplary material commonly fermented to form ethanol is corn. Corn oil is valuable for recovery. Other materials that can be fermented to produce ethanol and contain a desirable amount of oil for recovery include wheat, rice, and oats.

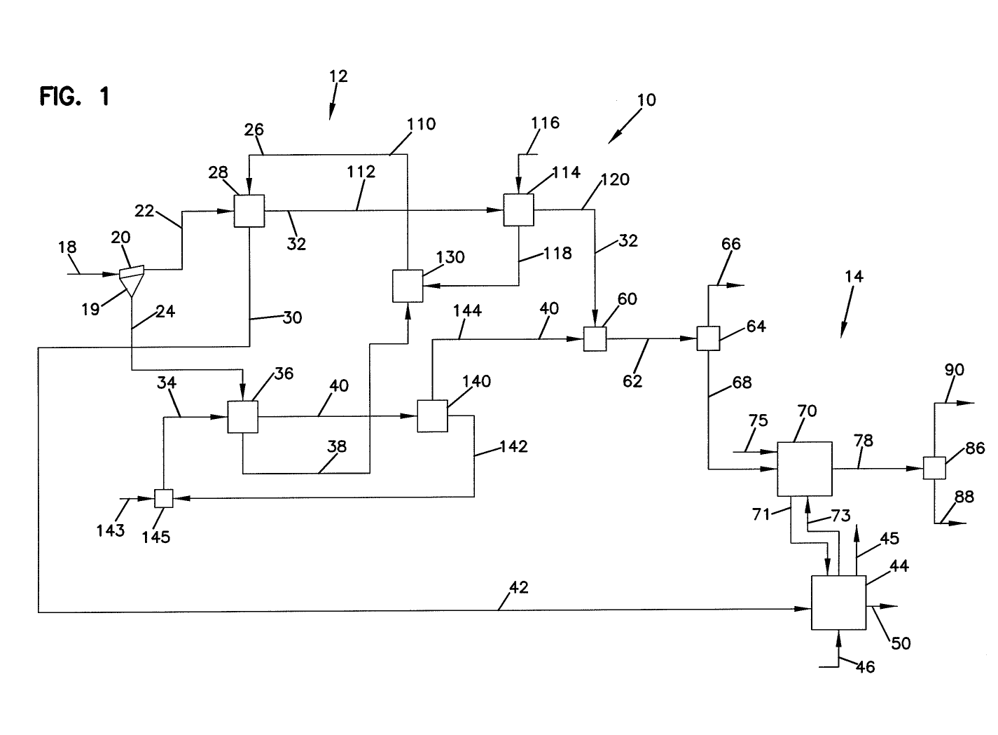

Now referring to FIG. 1, an alcohol production facility is schematically illustrated at reference number 10. The alcohol production facility 10 includes an oil from flour separation process 12 and a fermentation process 14. The oil from flour separation process 12 and the fermentation process 14 can be practiced together or separately, as desired. The oil from flour separation process 12 can be practiced independently from the fermentation process 14, and the fermentation process 14 can be practiced independently from the oil from flour separation process 12. Furthermore, both processes can occur at the same location or at different locations.

Now referring to the oil from flour separation process 12, flour 18 is provided to a separator 19 for separation into a first stream containing coarse flour 22 and a second stream containing fine flour 24. The separator 19 can be a screener 20. The first stream containing coarse flour 22 can be referred to more simply as the coarse flour, and the second stream containing fine flour 24 can be referred to more simply as the fine flour. The coarse flour 22 can be extracted with a first extraction solvent 26 in a first extractor 28. The first extractor 28 can be provided as a countercurrent percolation style extractor. As a result of extraction, oil moves from the coarse flour 22 into the first extraction solvent 26 thereby forming a first oil enriched extraction solvent 30 and an oil depleted coarse solids 32. Similarly, the fine flour 24 can be extracted with a second extraction solvent 34 in a second extractor 36. The second extractor 36 can be a stirred tank with a recycle pump. As a result of extraction, oil moves from the fine flour 24 to the second extraction solvent 34 thereby forming a second oil enriched extraction solvent 38 and an oil depleted fine solids 40.

The first oil enriched extraction solvent 30 can be referred as the first miscella. The second oil enriched extraction solvent 38 can be referred as the second miscella. In general, the term “miscella” refers to the combination of extraction solvent and oil. One technique for recovering oil from a miscella includes vaporizing the extraction solvent from the miscella. In the oil from flour separation process 12, the miscella stream 42 can be thermally treated in an oil concentrator 44. In general, the oil concentrator 44 can operate as a result of application of steam 46 thereto to drive off the extraction solvent as a solvent vapor 45. The solvent vapor 45 can be recovered and used in subsequent extraction. An alternative or supplementary heat source 71 can be provide to the oil concentrator 44 from, for example, the fermentation system 70. By way of example, the alternative or supplemental heat source 71 can be provided as ethanol vapor and, upon condensation, can be returned to the fermentation system as liquid ethanol 73. The alternative or supplemental heat source 71 can be used to offset or reduce amount of energy applied to the oil concentrator 44 via the steam line 46. Crude oil 50 can be recovered from oil concentrator 44. The crude oil 50 can be processed for the removal of impurities, if desired.

In the diagram shown in FIG. 1, the miscella stream 42 is depicted as a single line feed to the oil concentrator 44. If desired, the first miscella 30 and the second miscella 38 can both be feed directly to the concentration 44. In the diagram shown, the first miscella 30 forms the miscella stream 42, and the second miscella 38 is mixed with a washer miscella stream 118 in a miscella mixer 130 and forms the first extraction solvent 26. It should be understood that the arrangement of the various miscella or extraction solvent flows can be established to take advantage of the concentration of oil in the miscella. For example, if there is a relatively small amount of oil in an extraction solvent, that combination can be referred to as “partial miscella” or more simply as a “miscella” and can be used as an extraction solvent to pull more oil therein. In the context of this disclosure, the reference to an “extraction solvent” does not preclude the presence of oil therein. There is no requirement that an extraction solvent is pure (meaning that it contains no oil). Of course, the extraction solvent can be provided as pure for any extraction process, if desired.

The oil depleted coarse solids 32 and the oil depleted fine solids 40 can be recovered and used in downstream applications. For example, the oil depleted coarse solids 32 and the oil depleted fine solids 40 may contain nutritional value, and can be used as animal feed. Furthermore, the oil depleted coarse solids 32 and the oil depleted fine solids 40 may contain a starch or sugar value and can be used as a raw material for alcohol production. In either case, it is desirable to remove the extraction solvent therefrom. The oil depleted coarse solids 32 and the oil depleted fine solids 40 can be combined at a mixer 60 to form oil depleted solids 62. The oil depleted solid 62 can be dried in a dryer 64. From the dryer 64, extraction solvent vapor 66 can be recovered and used in subsequent extraction, and dry oil depleted solids 68 can be recovered.

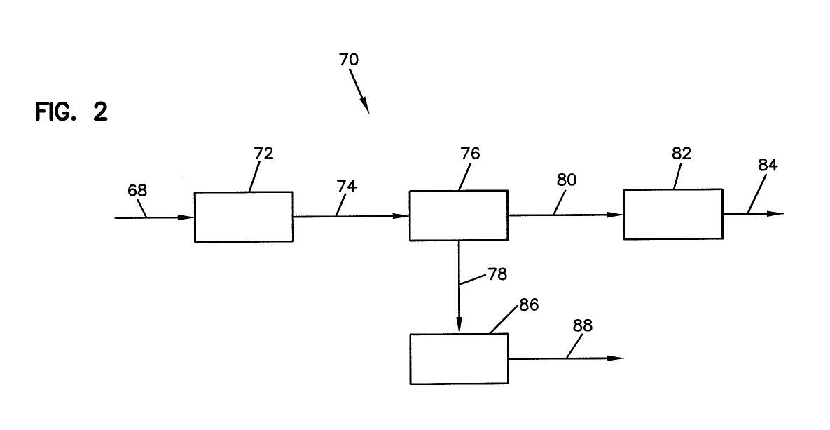

Now referring to the fermentation process 14, the dry oil depleted solids 68 can be combined with yeast and water and fermented in a fermentation system 70. Now referring to FIG. 2, an exemplary fermentation system 70 is depicted in diagrammatic form. The dry oil depleted solids 68 can be feed to a fermentation tank 72 where the dry oil depleted solids 68 are combined with water and yeast and allowed to ferment to form a beer stream 74 that can be feed to a beer column 76 where the beer stream 74 is separated into a whole stillage stream 78 and an aqueous alcohol stream 80. The aqueous alcohol stream 80 can be processed in a distillation and sieve system 82 to purify the alcohol and provide concentrated alcohol 84. The whole stillage 78 can be processed in a solids processing group of equipment 86 for the formation of dried deoiled distillers grain 88. In FIG. 1, the whole stillage 78 is shown leaving the fermentation system 70 and processed by the solids processing group of equipment 86 to form water vapor 90 and dried deoiled distillers grain 88.

The fermentation system 70 can generate vapor ethanol 71 (“190 vapors”) as a result of distillation. The ethanol vapor 71 can be used an alternative or supplemental heat source for driving the oil concentrator 44, and liquid ethanol 73 can be returned to the fermentation system 70. The fermentation system 70 can be driven by a heat source such as a steam line 75. It should be understood that the fermentation system is shown in abbreviated form and one skilled in the art would understand how the fermentation system can be operated in order to produce alcohol and dried deoiled distillers grain. The dried deoiled distillers grain can be characterized as having a low fat content (resulting in enhancing shelf life) and a high protein content. The dried deoiled distillers grain can be a desired animal feed product.

Referring again to the oil from flour separation process, the flour 18 is separated into a coarse flour stream 22 and a fine flour stream 24 in order to enhance the extraction process. The fines in certain types of flour have a tendency to cause clogging in commonly used extraction equipment. The fines can cause a coating of screens in commonly used extraction equipment, and the coating can result in plugging or clogging thereby making the extraction process more complicated and more expensive. By screening the flour, the flour can be separated into at least the two streams that can be handled separately for the extraction of oil therefrom. The coarse flour stream 22 can be characterized as a fraction that drains well when contacted with an extraction solvent in a extractor. As a result, by separating the fines from the coarse flour stream 22, oil can be relatively efficiently extracted from the coarse flour stream 22. The fine flour stream 24 can be characterized as a fraction that clogs a 40 mesh screen when contacted with extraction solvent in an extractor. In general, the screener 20 can provide a split based upon a 30 mesh screen. When corn flour is the flour 18, the split between fractions is expected in the range of about 3:7 to about 7:3, or about 4:5 to about 5:4. The fine flour stream 24 can be material that passes through a screen and has a size of less than about 600 μm and preferably less than about 400 μm. The course flour stream 22 can include material having a particle size nominally greater than 600 μm.

The coarse flour 22 is fed to a first extractor 28 and is contacted with a first extraction solvent 26. The first extraction solvent 26 can be provided as a partial miscella 110. In general, the term “miscella” refers to a combination of extraction solvent and oil. The reference to “partial miscella” simply means that the extraction solvent contains oil but still can be advantageously used as an extraction solvent in an extraction process. Using a partial miscella, when appropriate, as an extraction solvent can help decrease the cost of the process because using an extraction solvent that is completely free of oil may be generally more expensive than using an extraction solvent that has not been entirely purified of oil.

The coarse solids 112 from the first extractor 28 can be fed to a washer 114. In general, the washer 114 can be considered a type of extractor where extraction solvent 116 is combined with the coarse solids 112 resulting in a washer miscella 118 and washer solids 120. The washer solids 120 can be provided as the oil depleted coarse solids 32. The operation of the extractor 28 and the washer 114 can be fairly convenient because of the removal of the fines from the flour 18.

The washer miscella 118 can be combined with the second miscella 38 in the mixer 130, and can form the first extraction solvent 26 (and can be referred to as the partial miscella 110).

The oil depleted fine solids 40 from the second extractor 36 can be further processed in a centrifuge 140 for the separation of centrifuge miscella 142 from the fine solids 144. Second extractor miscella 38 can be recovered from the second extractor 36. In general, settled solids from the bottom of the second extractor 36 can be pumped out to form the oil depleted fine solids 40 and sent to the centrifuge 140. The second extractor miscella 38 can be provided as an overflow from the second extractor 36, and can be considered clarified miscella. A more complete separation between the miscella and the fine solids 40 can be provided by the centrifuge 140. Because of the separation of the flour 18 into a first stream containing coarse flour 22 and the second stream containing fine flour 24, the oil depleted fine solids 40 can be provided in a pumpable form. That is, the oil depleted fine solids 40 can be pumped and thereby conveyed from the second extractor 36 to the centrifuge 140. The centrifuge miscella 142 can be recycled and used as the second extraction solvent 34 in the second extractor 36. In addition, the centrifuge miscella 142 can be combined with extraction solvent 143 in the centrifuge miscella mixer 145. The amount of the extraction solvent 143 added to the centrifuge miscella 142, or the ratio of extraction solvent 143 to centrifuge miscella 142, can be controlled to provide the second extraction solvent 34 with a desired low concentration of oil. The extraction solvent 143 can be provided as pure extraction solvent which means that it contains no oil. Alternately, the extraction solvent 143 can contain oil.

In general, the flour can be combined with the extraction solvent at a weight ratio that provides a desired level of separation of oil from the flour into the extraction solvent. The weight ratio can vary for each of the extraction processes, such as the extractor 28, the washer 114, and the extractor 36. By way of example, for each extractor, the weight ratio of flour to extraction solvent can be about 0.5:1 to about 4.0:1.

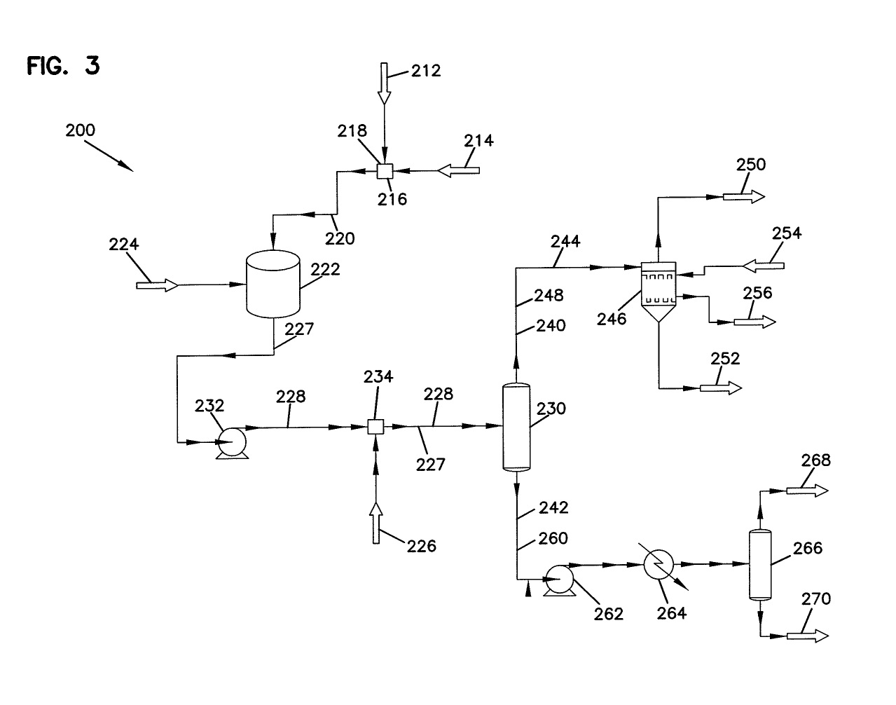

Now referring to FIG. 3, an alternative process for separating oil from an oil containing non-fermented vegetable material is schematically illustrated at reference number 200. The process 200 provides for the recovery of oil from an oil containing non-fermented vegetable material 212 that can be referred to more simply as the oil containing material 212. The oil containing material 212 can be provided as a solid. In general, the term “solid” refers to a material that does not flow like a liquid would flow. The characterization of the oil containing material as a solid does not mean that the oil containing material contains no water. In fact, it is expected that many solid oil containing materials (e.g., corn) contain water such as, for example, bound water. Furthermore, the solid material can be ground up or crushed so that it is in a form that flows. The material 212 can be provided as coarse flour 22, fine flour 24, or a combination of coarse flour 22 and fine flour 24. The process 200 can be provided as a continuous process or as a batch process.

The vegetable source 212 can be ground, cracked, milled, or otherwise processed to increase its surface area, and combined with an extraction solvent 214 at a mixing location 216 to form a crude mixture 218. There is no requirement that the vegetable source 212 is subjected to grinding, cracking, milling, or processing to increase its surface area. It is generally found, however, that increasing the surface area of the vegetable source 212 provides greater opportunity for the extraction solvent 214 to solubilize oil contained within the vegetable source 212. The mixing location 216 can be provided as a location where the vegetable source 212 and the extraction solvent 214 come together. The mixing location 216 can be provided as, for example, a static mixer, an in-line mixer, a tank, a stirred or mixed tank, or a series of cascading tanks In general, it is desirable for the oil containing material 212 and the extraction solvent 214 to sufficiently mix so that the extraction solvent 214 can extract oil from the oil containing material 212.

The crude mixture 218 flows through a crude mixture conduit 220 and into a mixing tank 222. The purpose of the mixing tank 222 is to provide the crude mixture 218 with residence time in order to allow the extraction solvent to contact the oil containing material and extract oil therefrom. In general, the length of the residence time depends on a number of factors including the ratio of the extraction solvent to oil containing material, the extraction solvent used, the oil containing material used, the amount of oil in the oil containing material, the temperature of the system, etc. In general, however, it is generally desirable to provide a residence time of at least about 10 minutes in order to provide the extraction solvent with sufficient time to interact with the solid material. Preferably, the residence time is at least about 15 minutes. In addition, the residence time can be limited in order to provide a process that is sufficiently fast. For example, the residence time can be less than about 45 minutes and preferably less than about 40 minutes. In should be understood, however, that the residence time can be any amount of time that provides a desired separation of oil from the vegetable source.

The ratio of the extraction solvent 214 to the oil containing material 212 can be selected to provide a desired speed and level of extraction of the oil from the oil containing material 22 and into the extraction solvent 214. In many application, the ratio of the extraction solvent 214 to the oil containing material 212 can be a weight ratio of about 0.5:1 to about 4:1, and preferably about 1:1 to about 3:1.

Once the crude mixture 218 has been provided with a sufficient residence time to allow for the extraction solvent to solubilize at least a portion of the oil in the oil containing material, the extraction solvent can be separated from the material and the oil can be recovered from the extraction solvent. In order to facilitate or enhance the separation of the extraction solvent from the material, water can be combined with the crude mixture 218. A water stream 224 can be added to the mixing tank 222, a water stream 226 can be added downstream of the mixing tank 222, or both a water stream 224 and a water stream 226 can be introduced. In the case of a batch operation, it may be desirable, for example, to introduce the water stream 224 to the mixing tank 222 after a sufficient residence time has been provided. The resulting multiple phase mixture 227 can be fed via conduit 228 to a settling tank 230. If desired, a pump 232 can be used to facilitate movement of the multiple phase mixture 227 through the conduit 228. In the case of a continuous operation, it may be desirable to introduce the water stream 226 to the solid material and organic solvent mixture 218 at a mixing location 234. When a water stream 226 is introduced at the mixing location 234, the optional pump 232 can be used to transport the crude mixture 218 through the conduit 228. The reference to a multiple phase mixture 227 is not intended to require the existence of separate phases but rather to characterize a mixture that has a tendency to phase separate if allowed to settle.

The amount of water added to the crude mixture 218 can be selected to provide a desired separation of the oil enriched extraction solvent from the oil depleted vegetable source (e.g., the oil depleted oil containing material), and to provide the water phase with a sufficient amount of water for further downstream processing. The amount of water can be selected to provide the multiple phase mixture 227 with a weight ratio of the crude mixture 218 to water of about 0.5:1 to about 4:1, and preferably about 1:1 to about 3:1.

The settling tank 230 is provided to allow for separation of the multiphase mixture 227 into an organic phase 240 and an aqueous phase 242. In many systems, it is expected that the organic phase 240 will be lighter than the aqueous phase 242 and, therefore, the organic phase 240 will rise to the top of the settling tank 230 and the aqueous phase 242 will sink to the bottom of the settling tank 230. It should be understood, however, that variations may exist. That is, in certain circumstances, the aqueous phase may rise to the top and the organic phase may sink to the bottom of the settling tank. In the process 200, the organic phase 240 is recovered via the organic phase conduit 244, and fed to an evaporator 246. The organic phase 40 can be referred to as a purified mixture 248. The introduction of the water facilitates the separation of the extraction solvent and oil solublized therein from the particulates that have a tendency to remain with the water phase 242. The evaporator 246 provides for separation of the extraction solvent from the oil. Accordingly, the extraction solvent 250 can be recovered and the crude oil 252 can be recovered. The extraction solvent 250 can be provided as a vapor, and condensed and recycled. The crude oil 252 can be processed for purification.

The evaporator 246 can be powered by any desired heat source for the separation of the extraction solvent from the oil. For example, steam 524 can be introduced into the evaporator 246 for driving the separation, and a condensate 256 can be recovered.

The aqueous phase 242 is allowed to flow through the aqueous phase conduit 260. Optionally, a pump 262 can be provided, a heater 264 can heat the aqueous phase, and a flash distillation operation 266 can be used to drive off residual extraction solvent 268. The remaining aqueous phase 270 can be provided as a wet solids material. An advantage of the wet solids material 270 is that it is in a form readily available for production of ethanol by fermentation.

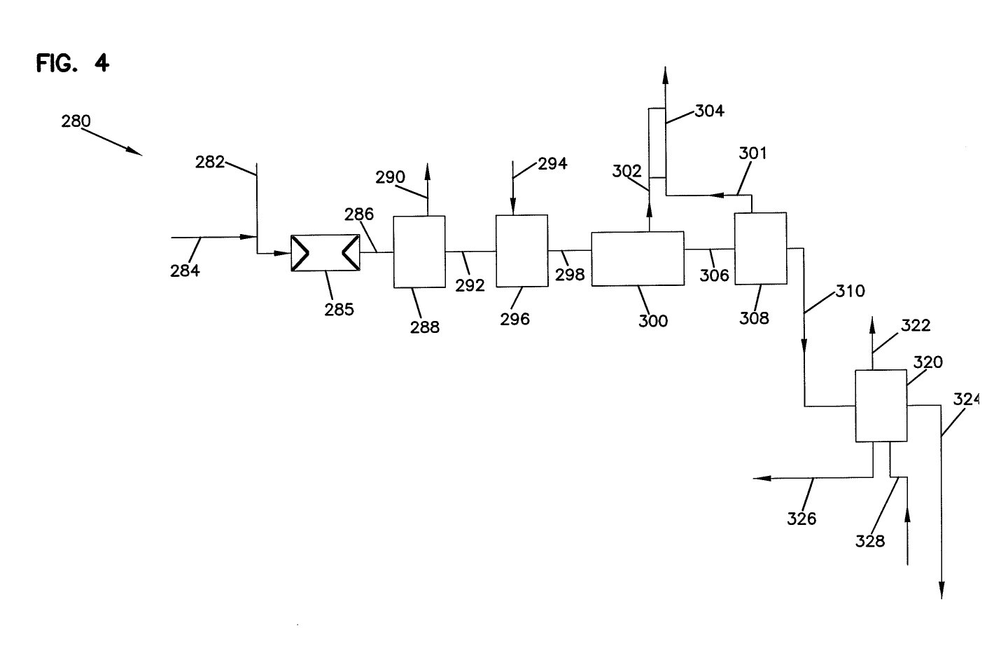

Now referring to FIG. 4, a process for the production of ethanol utilizing a wet solids material 282 is shown at reference number 280. The wet solids material 282 can be the aqueous phase 270 resulting from the process of FIG. 3. The wet solids material 282 can be characterized as a material containing a sugar source for fermentation and water, and wherein the material has been processed for the removal of oil therefrom. The wet solids material 282 can be provided having a water content of about 20 wt. % to about 35 wt. %. If desired, the water content of the wet solids material 282 can be increased by adding water 284. In the production of ethanol, it is desirable to heat the wet solids material 282 in a cooker 285. After heat treatment in the cooker 285, the cooked solids material 286 can be processed through a flash of evaporator 288 for the removal of volatiles 290 that may include residual extraction solvent. The cooked and volatilized solid materials 292 can be combined with enzymes 294 in a mixing tank 296 to form a liquid mash 298. The liquid mash 298 can be feed to a fermentation vessel 300 where the liquid mash 298 undergoes fermentation. Carbon dioxide 302 produced as a result of fermentation can be purged from the fermentation vessel 300 and processed through a scrubber 304. The resulting fermented product can be referred as a beer stream 306, and can be received in a beer well 308. The beer well 108 is generally provided as a storage area for receiving the beer stream 306 from the fermentation vessel 300, and providing a continuous source of beer 310 for feeding to a distillation system 320. Carbon dioxide 301 can be produced as a result of continued fermentation in the beer well 308 or can be liberated from the beer in the beer well 108, and can be recovered and processed via the scrubber 304.

The beer 310 is feed to the distillation system 320 for the recovery of alcohol. In general, the distillation system 310 results in an overhead, alcohol stream 322, a water stream 324, and a stillage stream 326. The distillation system 310 can be powered by introducing steam 328.

The foregoing description, which has been disclosed by way of the above discussion and the drawings, addresses embodiments of the present disclosure encompassing the principles of the present invention. The methods maybe changed, modified and/or implemented using various types of equipment and arrangements. Those skilled in the art will readily recognize various modifications and changes which maybe made to the described methods and equipment without strictly following the exemplary embodiments illustrated and described herein, and without departing from the scope of the present invention.

1. A method for separating oil from flour, the method comprising:
(a) separating flour into a first stream comprising coarse flour and a second stream comprising fine flour;
(b) combining a first extraction solvent with the first stream and extracting oil from the first stream to provide a first miscella and coarse solids;
(c) combining a second extraction solvent with the second stream and extracting oil from the second stream to form a second miscella and fine solids; and
(d) recovering oil from the first miscella and the second miscella.

2. A method according to claim 1, wherein the flour comprises an oil containing vegetable material.

3. A method according to claim 2, wherein the oil containing vegetable material comprises seed.

4. A method according to claim 3, wherein the oil containing vegetable material comprises corn.

5. A method according to claim 1, wherein the first stream comprising fine flour includes a screened material having a size of less than about 600 μm.

6. A method according to claim 1, wherein the first stream comprising fine flour includes a screened material having a size of less than about 400 μm.

7. A method according to claim 1, wherein the first extraction solvent and the second extraction solvent comprise the same organic solvent.

8. A method according to claim 1, wherein the first extraction solvent and the second extraction solvent are each selected from the group consisting of alkanes, alkenes, alcohols, ethers, aldehydes, aromatic hydrocarbons, and mixtures thereof.

9. A method according to claim 1, wherein the first extraction solvent and the second extraction solvent comprise hexane.

10. A method according to claim 1, wherein the steps of extracting comprise combining the flour with the extraction solvent at a weight ratio of the flour to the extraction solvent of about 0.5:1 to about 4.0:1.

11. A method according to claim 1, further comprising combining the coarse solids and the fine solids to form deoiled solids.

12. A method according to claim 11, further comprising removing the first extraction solvent and the second extraction solvent from the deoiled solids to form dried deoiled solids.

13. A method according to claim 12, further comprising fermenting the dried deoiled solids for the production of ethanol.

14. A method according to claim 1, wherein the steps of recovering the oil from the first miscella and the second miscella comprises vaporizing the first extraction solvent from the first miscella and vaporizing the second extraction solvent from the second miscella.

15. A method according claim 1, wherein the step of combining a second extraction solvent with the second stream and extracting oil from the second stream to form a second miscella and fine solids comprises:
(a) extracting oil from the second stream into the second extraction solvent in an extractor; and
(b) recovering the second miscella as a result of centrifuging a stream from the extractor.

16. A method according claim 15, further comprising recovering the second miscella from the extractor prior to centrifuging.

17. A method according to claim 1, wherein at least about 50% of the oil in the flour is extracted as a result of the extraction steps.

18. A method according to claim 1, wherein the step of combining a first extraction solvent with the first stream and extracting oil from the first stream to provide a first miscella and coarse solids comprises:
(i) providing a mixture of the first stream and the first extraction solvent and extracting oil from the first stream with the first extraction solvent;
(ii) combining the mixture with water and forming an organic phase and a water phase; and
(iii) separating the organic phase and the water phase to form:
A) the organic phase comprising oil enriched water immiscible extraction solvent;
and
B) the water phase comprising water and oil depleted coarse solids.

19. A method for producing ethanol comprising:
(a) recovering the coarse solids and the fine solids from the process of claim 1 to form dried deoiled solids; and
(b) fermenting the dried deoiled solids.

20. A method according to claim 19, wherein the first extraction solvent and the second extraction solvent are each selected from the group consisting of alkanes, alcohols, alkenes, ethers, aldehydes, aromatic hydrocarbons, and mixtures thereof.

21. A method according to claim 19, wherein the first extraction solvent and the second extraction solvent are each comprise hexane.

22. A method according to claim 19, wherein the step of combining the oil containing material with the extraction solvent comprises combining the flour with the extraction solvent at a weight ratio of the flour to the extraction solvent of about 0.5:1 to about 4.0:1.

23. A method according to claim 19, wherein the step of combining a second extraction solvent with the second stream and extracting oil from the second stream to form a second miscella and fine solids comprises:
(a) extracting oil from the second stream into the second extraction solvent in an extractor; and
(b) recovering the second miscella as a result of centrifuging a stream from the extractor.

24. A method according claim 23, further comprising recovering the second miscella from the extractor prior to centrifuging.

25-39. (canceled)
